(19) United States
(12) Patent Application Publication
(10) Pub. No.: US 2025/0241569 A1
(43) Pub. Date: Jul. 31, 2025

(54) BLOOD COLLECTION DEVICE

(71) Applicant: The University of Melbourne, Melbourne, Victoria (AU)

(72) Inventors: David John COLLINS, Melbourne, Victoria (AU); John Christopher KOUNETAS, Melbourne, Victoria (AU); Maciej NECKI, Melbourne, Victoria (AU); Luke Pasquale SAMMARTINO, Melbourne, Victoria (AU)

(21) Appl. No.: 18/699,932

(22) PCT Filed: Oct. 11, 2022
(86) PCT No.: PCT/AU2022/051222

§ 371 Date: Apr. 10, 2024

(30) Foreign Application Priority Data
Oct. 11, 2021 (AU) 2021903260

(51) Int. Cl. A61B 5/15

(52) U.S. Cl. CPC A61B 5/15003; A61B 5/150267; A61B 5/15074

(57) ABSTRACT

A blood collection device comprising a dispenser part having a plurality of independent outlet fluid passages, each terminating at a respective outlet port for coupling with a detachable storage container; and a receiver part having an inlet fluid passage associated with an inlet port for coupling with a blood transfer tube, the receiver part being operatively coupled with, and moveable relative to, the dispenser part so that the inlet fluid passage is moveable to align with any one of the outlet fluid passages to form a connected fluid pathway therebetween.

### FIELD OF THE INVENTION

This invention relates to a blood collection device, and in particular a multi-port device that is suitable for use in blood collection by passive venous pressure.

### BACKGROUND

Conventional methods of blood collection involve the use of elevated hydrostatic pressure differentials between a collection device (typically an evacuated container) and a patient's blood vessel to drive the flow of blood from the patient to the collection device. Once a connection is made between an evacuated container and a patient's blood vessel, via for example a piercing apparatus such as a butterfly needle and an associated fluid transfer tube, blood is rapidly drawn from the patient into the container owing to large vacuum-induced pressure forces.

A problem with such blood collection techniques is that large forces placed on the blood during the collection process can damage the blood cells (haemolysis) or lead to thrombogenesis, thereby rendering blood collected in this manner void for subsequent analyses or application. Additionally, conventional collection techniques as described waste significant volumes of blood as tens of millilitres of bloods are drawn per sample despite only drops being required for analyses. This aggregates to potentially litres of unused and discarded blood for patients who require frequent blood tests, such as those who are being closely monitored during an Intensive Care Unit (ICU) stay, and potentially causing iatrogenic anaemia.

Conventional blood collection processes may also be difficult to perform, typically by an individual healthcare practitioner without any peer assistance, on some parts of the population such as children and the elderly, particularly in instances where primary veins of the patient are difficult to access and when multiple containers of samples must be collected while seating. Further, drawing blood using vacuum pressure techniques from smaller veins and capillaries could be lengthy and potentially traumatic for the patient and risk contaminating collected samples.

Therefore, there exists a need for a solution that provides a safer, less wasteful and easier way to collect blood samples from patients. The solution should ideally be compatible with existing piercing apparatuses such as the butterfly needle assembly as well as conventional blood storage containers.

The applicant has determined that it would be advantageous to provide a blood collection device which, in its preferred embodiments, seeks to at least in part alleviate the above-identified problems or to offer the public with a useful choice.

### SUMMARY OF THE INVENTION

According to an aspect of the invention, there is provided a blood collection device comprising a dispenser part having a plurality of independent outlet fluid passages, each terminating at a respective outlet port for coupling with a detachable storage container; and a receiver part having an inlet fluid passage associated with an inlet port for coupling with a blood transfer tube, the receiver part being operatively coupled with, and moveable relative to, the dispenser part so that the inlet fluid passage is moveable to align with any one of the outlet fluid passages to form a connected fluid pathway therebetween.

In some embodiments, the dispenser part comprises an air channel located proximate each respective outlet port, the air channel being configured to form part of an airflow pathway between a storage container coupled to the respective outlet port and the ambient, during use.

In some embodiments, the receiver part is configured to be rotatable relative to the dispenser part.

In some embodiments, the receiver part is frictionally coupled to the dispenser part by way of a circumferential seal, and wherein the seal is a fluorocarbon type o-ring.

In some embodiments, the receiver part comprises a handle to assist a user with moving the receiver part relative to the dispenser part in use.

In some embodiments, the handle is ring shaped and/or integrally attached to the receiver part.

In some embodiments, the receiver part comprises a central cavity to allow passage of airflow.

In some embodiments, the central cavity is covered with a filter unit comprising an air-permeable filter material.

In some embodiments, an end of the inlet fluid passage opposing the inlet port comprises an outlet seal, and wherein the outlet seal is a fluorocarbon type o-ring.

In some embodiments, the receiver part is mounted within a cavity of the dispenser part.

In some embodiments, the dispenser part is in the form of a substantially round hub with downwardly-oriented outlet ports.

In some embodiments, the dispenser part comprises two, three or four outlet ports.

In some embodiments, each of the respective outlet ports comprises a part-side wall of slanted geometry to assist with removal of a docked storage container by a twist-release action.

In some embodiments, contact surfaces between the dispenser part and the receiver part are configured to be substantially smooth.

In some embodiments, the dispenser part comprises a circumferential wall with sideway projections proximate the location of each respective outlet port.

In some embodiments, the inlet port extends substantially upwardly from the receiver part.

In some embodiments, the air channel leads to the central cavity of the receiver part and the airflow pathway extends out of an opening of the receiver part.

In some embodiments, the receiving part is operatively coupled to the dispensing part by a plurality of prongs.

In some embodiments, one or more of said prongs are provided with a recess at its tip portion for receiving a corresponding lip projection of the dispensing part by frictional engagement.

In some embodiments, a gasket seal having a single opening for allowing fluid flow between the inlet fluid passage and the outlet fluid passage is provided between the receiving part and the dispensing part, when assembled.

According to a further aspect of the present invention, there is provided a blood collection system, comprising a blood collection device as described above; a butterfly needle connected to a blood transfer tube for coupling with the inlet port of the receiver part; and one or more detachable storage containers for coupling with the outlet port of the dispenser part.

In some embodiments, detachable storage containers are not vacuum-sealed or negative pressure containers.

In some embodiments, blood is collected from a patient during use of the system by passive venous pressure.

Further aspects of the present invention and further embodiments of the aspects described in the preceding paragraphs will become apparent from the following description.

While components of the blood collection device and system will be described below for use in combination with each other in the preferred embodiments of the present invention, it is to be understood by a skilled person that some aspects of the present invention are equally suitable to be used interchangeably between one or more embodiments of the present invention and/or suitable for use as standalone inventions that can be individually incorporated into other collection devices and assemblies not described herein.

The word “about” or “approximately” when used in relation to a stated reference point for a quality, level, value, number, frequency, percentage, dimension, location, size, amount, weight or length may be understood to indicate that the reference point is capable of variation, and that the term may encompass proximal qualities on either side of the reference point.

As used herein, the word “substantially” may be used merely to indicate an intention that the term it qualifies should not be read too literally and that the word could mean “sufficiently”, “mostly” or “near enough” for the patentee's purposes.

### DESCRIPTION OF THE DRAWINGS

The invention will now be described, by way of non-limiting example only, with reference to the accompanying drawings, in which:

FIG. 1 is a perspective schematic view of a blood collection system in accordance with a preferred embodiment of the present invention;

FIG. 2 is a perspective exploded view of a blood collection device in accordance with the preferred embodiment of the present invention;

FIG. 3 is an exploded sectional view of the blood collection device;

FIG. 4 is an assembled sectional view of the blood collection device coupled with storage containers;

FIG. 5 is a perspective view of the blood collection device without any coupled storage containers, tubing and needle;

FIG. 6 is a perspective schematic view of a blood collection system in accordance with another preferred embodiment of the present invention;

FIG. 7 is a perspective exploded view of the blood collection device of FIG. 6;

FIG. 8 is an exploded sectional view of the blood collection device of FIG. 6;

FIG. 9 is an assembled sectional view of the blood collection device of FIG. 6 coupled with storage containers;

FIG. 10 is a perspective view of the blood collection device of FIG. 9;

FIG. 11A is a perspective view from below of the blood collection device of FIG. 6 and FIG. 11B is a close up view of a lip connection part of the blood collection device of FIG. 11A;

FIG. 12 is a perspective view from below of a dispensing part of the blood collection device of FIG. 6;

FIG. 13 is a perspective view from below of a receiving part of the blood collection device of FIG. 6; and

FIG. 14 is a sectional perspective view of the blood collection device of FIG. 6 showing the airflow pathway.

### DETAILED DESCRIPTION

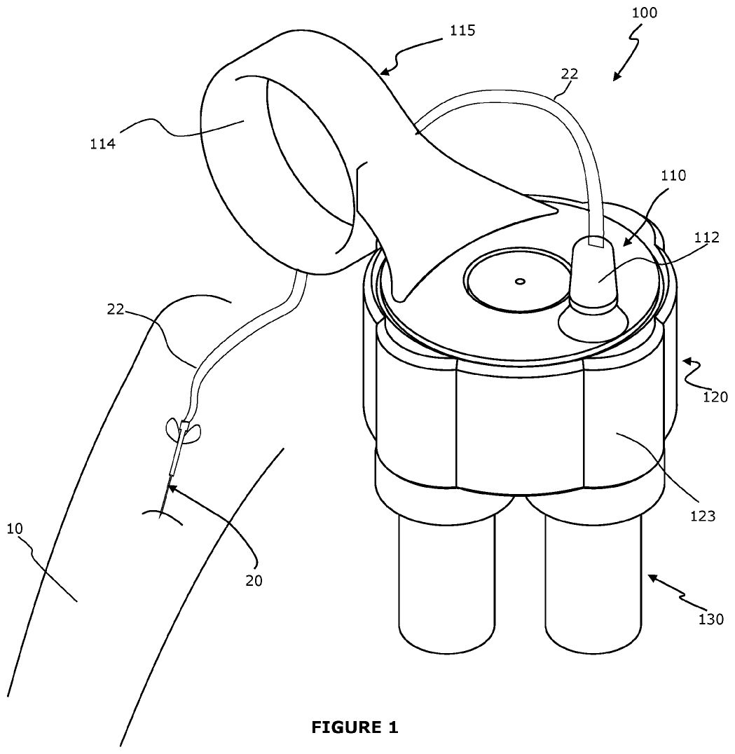

FIG. 1 shows a blood collection system comprising a blood collection device 100 according to a preferred embodiment of the present invention, a butterfly needle arrangement 20 which is configured for piercing tissues of a patient 10, a fluid transfer tube 22 connecting the butterfly needle arrangement 20 and the blood collection device 100, and a number of blood storage containers 130 for storing blood collected from the patient 10. It is to be understood that the blood collection device 100 is intended to be compatible with conventional medical piercing apparatuses such as the butterfly needle arrangement 20 example provided herein, as well as conventional blood storage containers 130 that are configured and dimensioned suitable for medical use.

Referring to FIGS. 1 to 4, the blood collection device 100 of the preferred embodiment comprises a two-part assembly which includes a receiver part 110 for interfacing with a fluid transfer tube 22 and a dispenser part 120 for channelling any fluid, such as blood, from the receiver part 110 out to detachable storage containers 130 coupled with the dispenser part 120 of the device 100 during use. In the preferred embodiment, the receiver part 110 is substantially donut shaped in the form of a cylinder 116 with a central cavity 141, and the receiver part 110 is designed to be received within a correspondingly shaped cavity of the dispenser part 120, which is itself shaped in the form of a substantially circular hub. It is to be understood that the shape and configurations of the receiver and dispenser parts 110, 120 described herein are provided as non-limiting examples of the present invention and that the parts 110, 120 could take on other shapes and forms that may be equally suitable for use in realising the advantages of the invention.

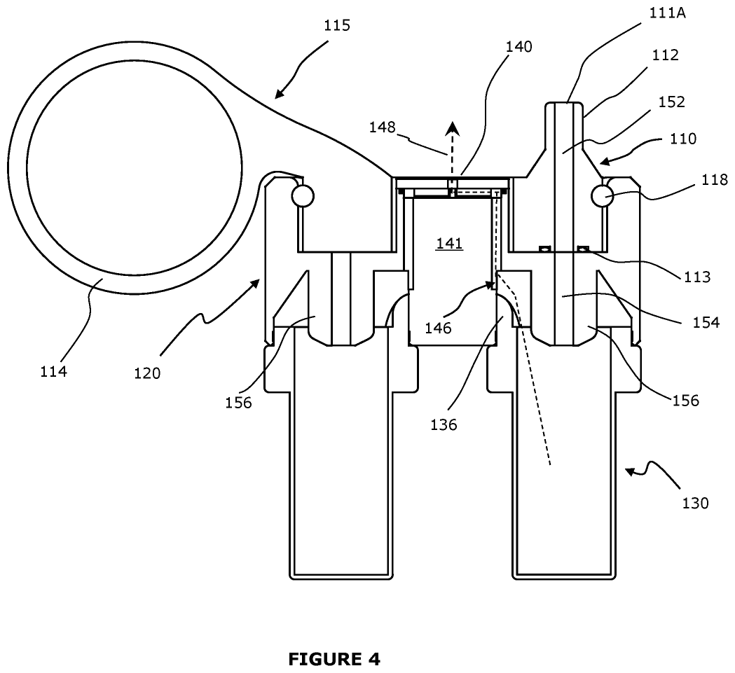

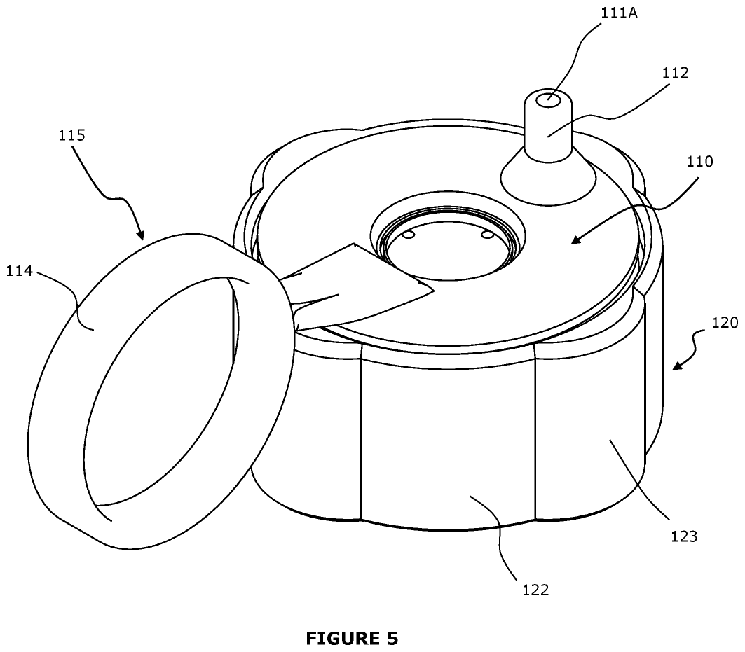

Turning to FIG. 4, the sectional assembled view of the blood collection device 100 shows the receiver part 110 being received in a topside cavity 121 of the dispenser part 120. In the preferred embodiment, the receiver part 110 is operatively coupled to the dispenser part 120 via frictional coupling or interference fit by way of a circumferential seal 118 in the form of an o-ring. It is preferred that the o-ring is constructed from a chemically-resistant fluorocarbon material. In the preferred embodiment, the bottom surface 119 of the receiver part 110 and the cavity surface 149 of the dispenser part 120 are configured to have a flush fit, when assembled. Preferably, the contact surfaces 119, 149 are machined to be substantially smooth to minimise any gaps or imperfections which could interrupt a sealed contact between the surfaces. This will reduce the risk of unwanted fluid leakage into or flowing between the mated surfaces 119, 149 when they are assembled in a flush fit.

In the preferred embodiment, the receiver part 110 is provided with a single inlet fluid passageway 152 that runs between a top opening 111A of an inlet port 112, which interfaces with a blood transfer tube 22, right through to a bottom opening 111B at the underside of the receiver part 110. The bottom opening 111B of the passageway 152 is configured to be substantially or wholly alignable with an opening 144A of any one of an outlet fluid passageways 154 of the dispenser part 120, which will be described below. An outlet seal 113 is provided at the bottom opening 111B of the inlet fluid passageway 152 to further reduce unwanted leakage of fluid between the mated surfaces 119, 149 during use. Additionally, the outlet seal 113 is configured to provide sufficient sealing pressure resistance to stop or partially stop any fluid flow between the inlet fluid passageway 152 and the outlet fluid passageway 154 when they are not aligned or partially-aligned. Preferably, the outlet seal 113 is in the form of an o-ring that is constructed from a chemically-resistant fluorocarbon material. In other embodiments, the o-ring is not required and instead the seal around the bottom opening 111B could be made from the same material and component as the dispenser part 120 or receiving part 110, and this does not need to be a separate seal component. In one embodiment, the inlet port 112 extends substantially upwardly from the receiver part for ease of connection with a fluid transfer tube 22.

In one embodiment, the receiver part 110 is provided with a circumferential groove 117 for locating the circumferential seal 118. The groove 117 and the seal 118 are suitably dimensioned to ensure an operationally sealed coupling between the receiver part 110 and the dispenser part 120, while allowing rotational movement of the receiver part 110 relative to the dispenser part 120 when so assembled.

In addition, the central cavity 141 of the receiver part 110 is configured to interface with a correspondingly dimensioned raised projection 124 of the dispenser part 120. The raised projection 124 has a top recess, which comprises openings 142 that each leads to a corresponding air channel 146 which will be described below. The central cavity 141 of the receiver part 110 in combination with the recess of the raised projection 124 provide each air chancel 146 a path for airflow to the ambient environment, when the device 100 is in the assembled state. In some embodiments, the cavity 141 of the receiver part is provided with a top cap 126 to enclose the cavity 141 while an opening 140 on the cap 126 allows airflow from the cavity 141 to the ambient. In other embodiments, the cavity 141 is configured to house a filter unit 127 which filters fluid or airflow from the cavity 141 to the ambient. The filter unit 127 may, in one example, comprise an air-permeable filter material.

The receiver part 110 further comprises a handle 115 for use by a healthcare practitioner to rotate the receiver part 110 relative to the dispenser part 120 when administering the blood collection device 100. In one embodiment, the handle 115 comprises a ring-shaped structure 114 that is integrally connected to a body of the receiver part 110. In other embodiments, the handle 115 may be configured to be removably attachable to a top or side portion of the receiver 110. While the handle 115 has been described to be a ring-shaped structure, which is easy to access by a user's finger or thumb, it should be understood that other handle structures not described herein may also be suitable without departing from the spirit of the invention.

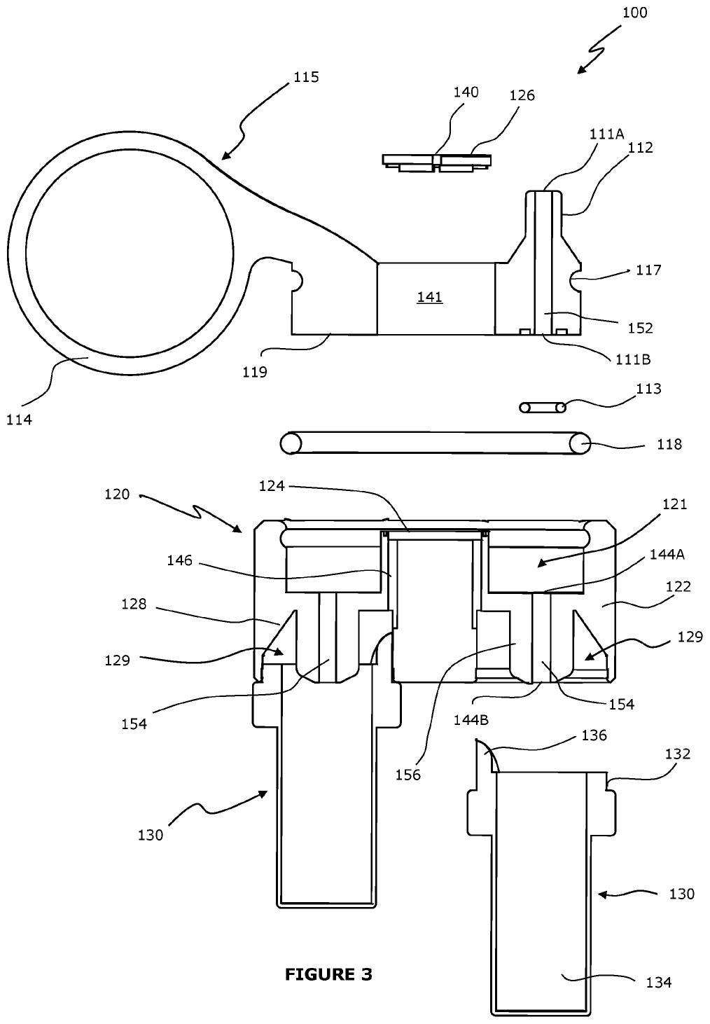

With reference to FIG. 3, the dispenser part 120 comprises a plurality of outlet fluid passageways 154 which extend between the top opening 144A and the bottom opening 144B of a dispensing member 156 of the dispenser part 120. In the preferred embodiment, the plurality of outlet fluid passageways 154 are said to be independently located, which is to say that the fluid passageways 154 are separated from one another and are not fluidically connected in any way so as to avoid cross contamination. Each of the outlet fluid passageways 154 terminates at a respective outlet port 129, which is shaped and configured to sealingly receive a detachable storage container 130 by coupling with a top portion 132 of the storage container 130. In the preferred embodiment, the respective outlet ports 129 are located towards a bottom side of the dispenser part 120, and preferably the outlet ports 129 themselves also substantially face the bottom of the dispenser part 120 so that any connected storage containers 130 are oriented substantially vertically in use. This advantageously allows the healthcare practitioner to readily follow any volume or fill level indicators located on a tube 134 portion of the container 130 as it is being filled.

The dispenser part 120 may be provided with any suitable number of outlet fluid passageways 154 and outlet ports 129, depending on the size and configuration of the blood collection device 100. In the preferred embodiments, the dispenser part 120 is provided with four outlet fluid passageways 154 and associated outlet ports 129 spaced around the dispenser part 120. In other embodiments, the dispenser part 120 is instead provided with three outlet fluid passageways 154 and outlet ports 129.

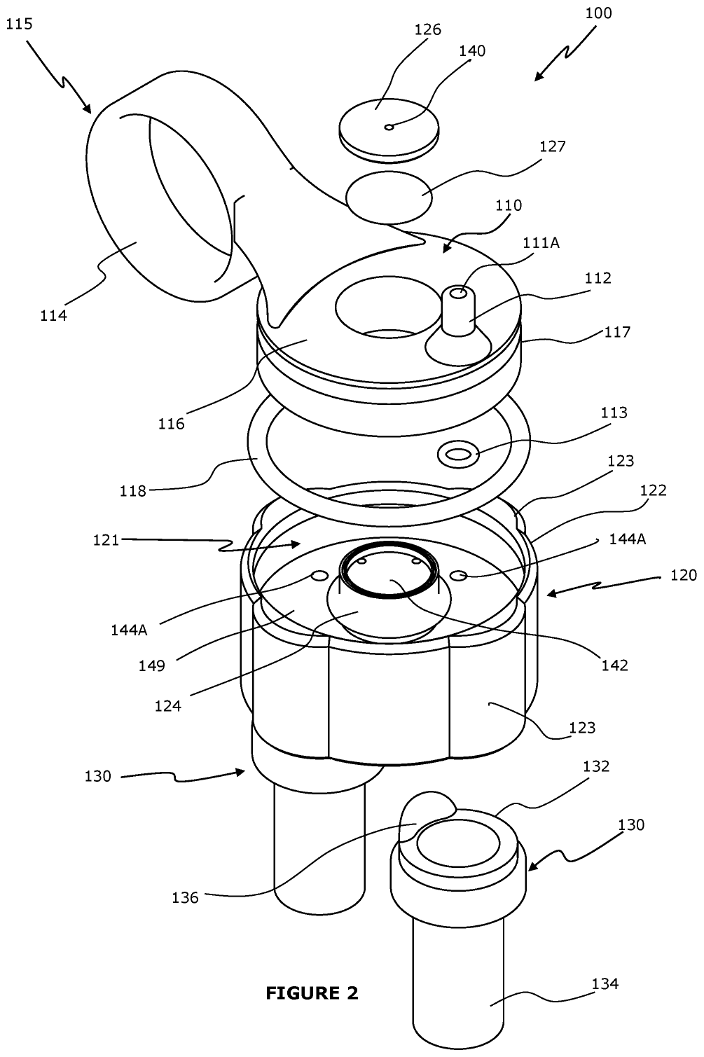

In the preferred embodiment, an air channel/opening 146 is provided in the dispenser part 120 at a location proximate each of the outlet ports 129 so as to form an airflow pathway 148 between an internal cavity of a detachable storage container 130 and said air channel 146 when the container 130 is coupled to the dispenser part 120 during use. This airflow pathway 148 allows air or any gaseous content to escape the internal cavity of the container 130 as it is being filled without causing a pressure build-up. The air channel 146 is in turn configured to terminate at the opening 142 of the dispenser part 120 as shown in FIG. 2. Gaseous fluid flow could then travel out to the ambient via the central cavity 141 of the receiver part 110 or opening 140 of the top cap 126, and optionally via the filter unit 127 as described above. In some embodiments, the air channel 146 may be dimensioned such that the Laplace pressure formed at the junction between any backflow of fluid (such as blood) collected in the container 130 and the air within the air channel 146 would be sufficient to prevent the flow of such fluid from flowing through the air channel 146 and out of the assembled collection device 100.

The complete airflow pathway 148 is formed from the internal cavity of the container 130, through the air channel 146 into the central cavity 141 of the receiver part 120, and then out to the ambient environment. In the preferred embodiment, this airflow pathway is available to each of the outlet ports 129, and to each the containers 130 when it is coupled to the respective outlet port 129. This feature advantageously allows the blood collection device 100 to work with non-vacuum based blood collection techniques such as collection methods that rely on passive venous pressure to drive blood flow from the patient 10 to the storage container 130. Passive venous pressure techniques typically involve much lower pressure forces (as it operates at a patient's normal blood pressure) than vacuum-based active pressure collection techniques, thereby reducing the risk of causing damage to the blood cells being collected during the process.

As described earlier, the two-part blood collection device 100 is configured so that the receiver part 110 is not only operatively coupled with the dispenser part 120, but also moveable relative to the dispenser part 120 in use such that the inlet fluid passageway 152 can be positioned to align with any one of the outlet fluid passageways 154 to form a connected fluid pathway therebetween. When the connected fluid pathway is formed between the inlet fluid passageway and one of the outlet fluid passageways, blood being collected from the patient 10 travels from the butterfly needle and transfer tube 22 through the connected fluid pathway and directly into the storage container 130.

In the preferred embodiment, the receiver part 110 is configured to be rotatable relative to the dispenser part 120, which is in the form of a round hub, so that a user may actuate the handle 115 to rotate the receive part 110 and, with it, the inlet fluid passageway 152 into direct or partial alignment with the outlet fluid passageway 154 to establish the connected fluid pathway for fluid collection. It is to be noted that while the circular movement and configuration of the receiver part 110 and the dispenser part 120 have been described as an exemplary example of the present invention, other shapes and relative movement configurations between a receiver part and dispenser part may also be suitable and workable without departing from the invention.

During use, once a storage container 130 has been filled using the described blood collection device 100, the user could simply rotate the receiver part 120 using the handle 115 to dis-align the inlet fluid passageway 152 with the outlet fluid passageway 154, which will disconnect the fluid pathway and stop further filling of the container 130. In other words, fluid is prevented from passing between the inlet fluid passageway 152 and the outlet fluid passage way 154 if the user dis-align them or move them to an in-between position to prevent overfilling of the container 130 or fluid spillage. If additional container 130 require filling, then the user may operate handle 115 to rotate the receiver part 110 again to align the inlet fluid passageway 152 with a different outlet fluid passageway 154 so as to fill another coupled container 130. The operating process of rotating the receiver part 110 of the device 100 using the handle 115 greatly simplifies the blood collection process for the medical practitioner and advantageously allows the practitioner to operate the blood collection device with only a single hand while leaving another hand free to assist the patient. In the preferred embodiment, the handle 115 and the circumferential seal 118 are configured to sustain the weight of the assembled device 100 and any attached containers 130 so that the handle 115 not only acts as an ergonomic tool for rotating the receiver part 110 but also as a primary weight-bearing gripping structure for handling the device 100.

The outlet ports 129 of the device is designed to be compatible with conventional storage containers 130 of various sizes and geometries. Containers 130 with smaller volume capacities may also be used to reduce the collection of unused blood from the patient. In some embodiments, each of the outlet ports 129 is also configured with a part-side wall 128 of slanted geometry to assist with removal of a docked storage container 130 by a twist-release action. In use, the user may twist the container 130 so that a raised portion 136 abuts the slanted wall 128 so as to push and release the container 130 from sealed coupling with the respective outlet port 129.

In some embodiments, a circumferential wall 122 of the dispenser part 120 may be configured with sideway projections 123 or bulges to account for the shape and size of the outlet port 129 and containers 130, and to assist with the handling and gripping of the device 100 in the user's hand. In the embodiments described, the sideway projections 123 are located proximate each respective outlet port 129. In other embodiments, the projections 123 may be configured with indicators/labels or colours to identify the order/size/type of specific outlet ports 129 or connected containers 130 so as to allow for case of identification and order of operation (e.g. rotating the receiver part 110 clockwise or counter-clockwise relative to the dispenser unit 120). It is to be appreciated that, in the preferred embodiment, the receiver part 110 is configured to move relative to the dispenser unit 120 in both clockwise and counter-clockwise directions.

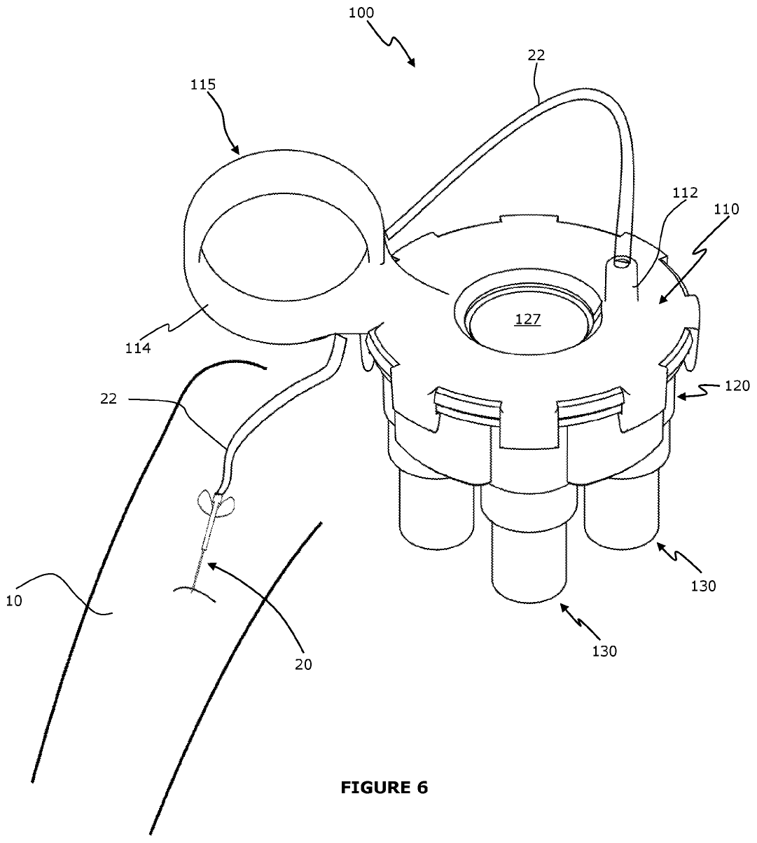

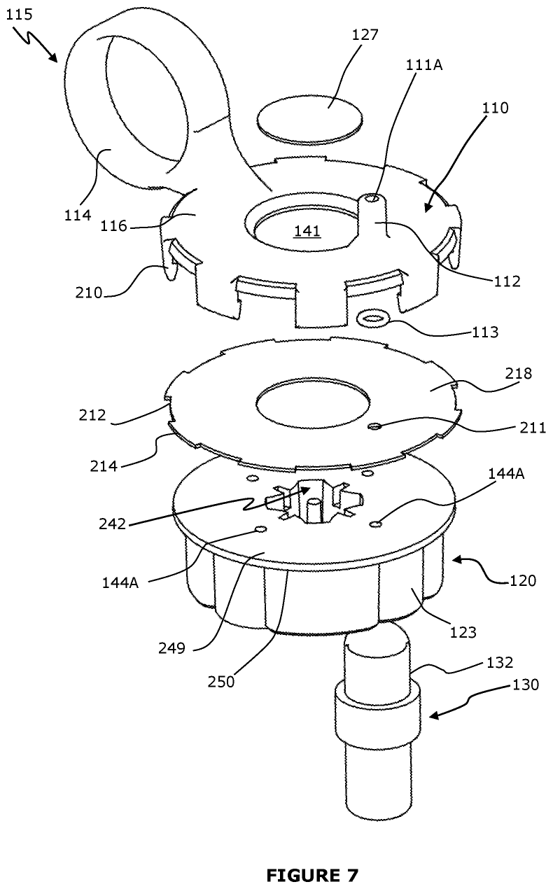

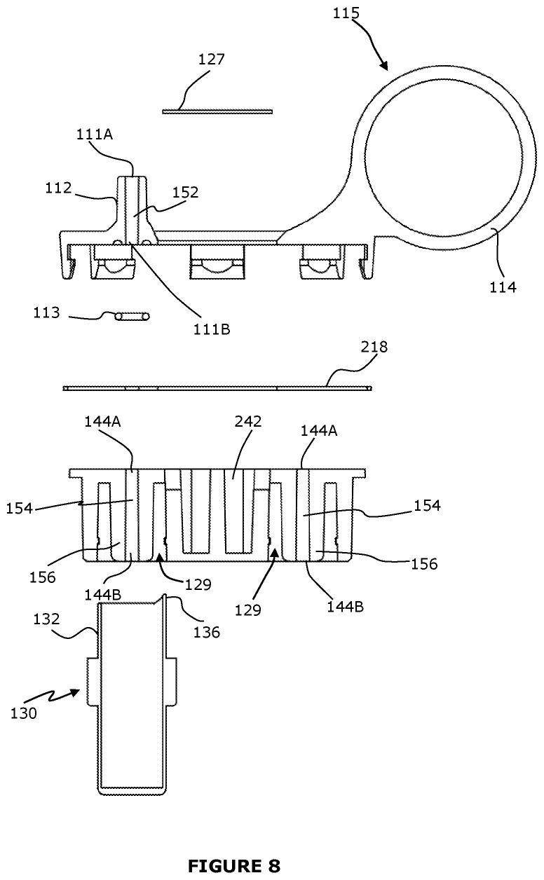

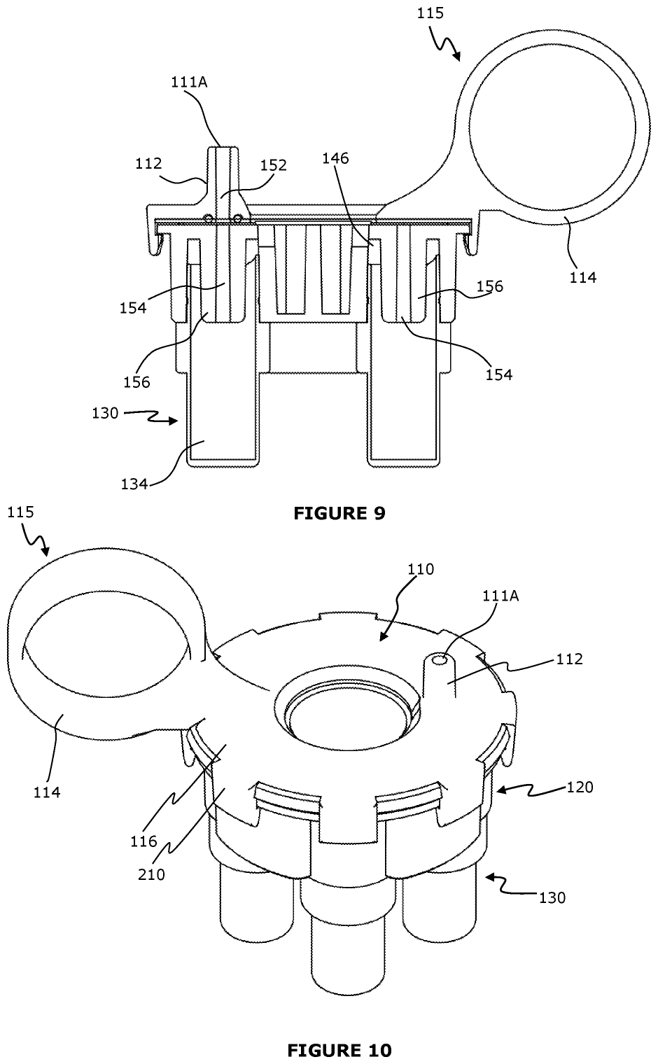

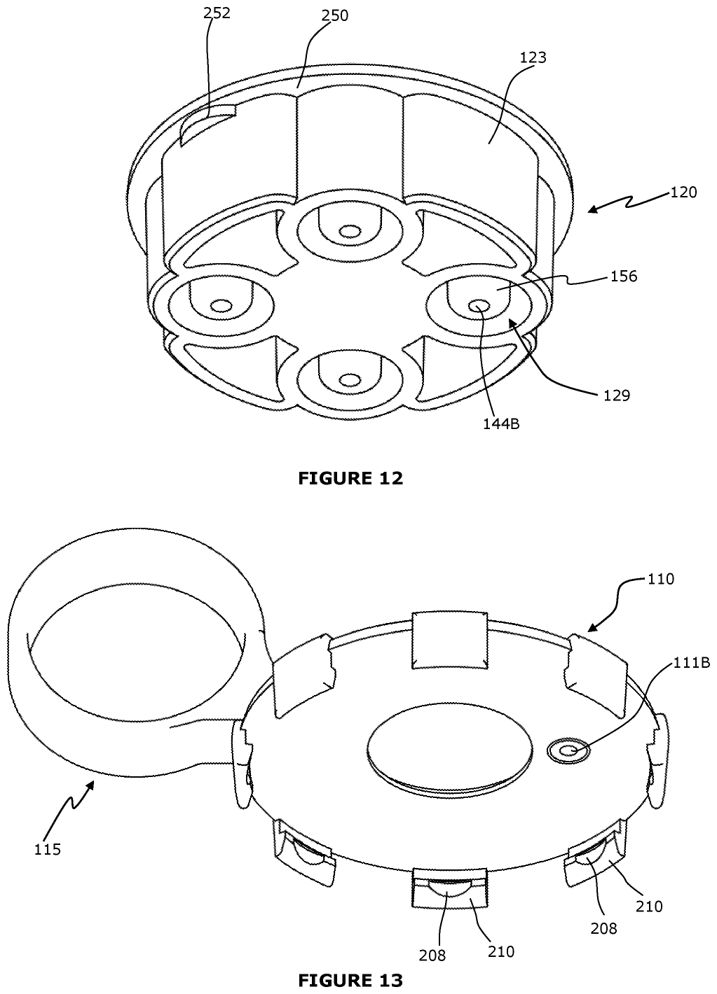

A further preferred embodiment will now be described with reference to FIGS. 6 to 14. In the description and drawings of this and other embodiments, same reference numerals are used as have been used in respect of the first embodiment, to denote and refer to corresponding features. In the further preferred embodiment as seen in FIG. 7, the receiving part 110 has been modified to comprise a number of prongs 210 for frictional coupling with a corresponding lip portion 250 of the dispensing part 120. Instead of having a large cavity 121 in the dispensing part 120 of the earlier described embodiment, the dispensing part 120 of this embodiment is provided with a substantially flat top surface 249 and a smaller internal air outlet cavity 242. A gasket 218 is provided instead of the o-ring 118 for sealing surfaces between the receiving part 110 and the dispensing part 120. The gasket 218 comprises a single opening 211 for allowing fluid flow between the inlet fluid passageway 152 of the receiving part 110 and outlet fluid passageway 154 of the dispensing part 120. The gasket 218 is also provided with alternating projections 214 and notches 212 at its perimeter to align with geometries of the prongs 210 so that the gasket rotates with the rotational movement of the prongs 210 as the user turns the receiving part 110 using the handle 115. In some embodiments, an o-ring seal 113 can be positioned at the bottom opening 111B of the inlet fluid passageway 152, while in other embodiments this o-ring may not be required due to presence of the gasket seal 218.

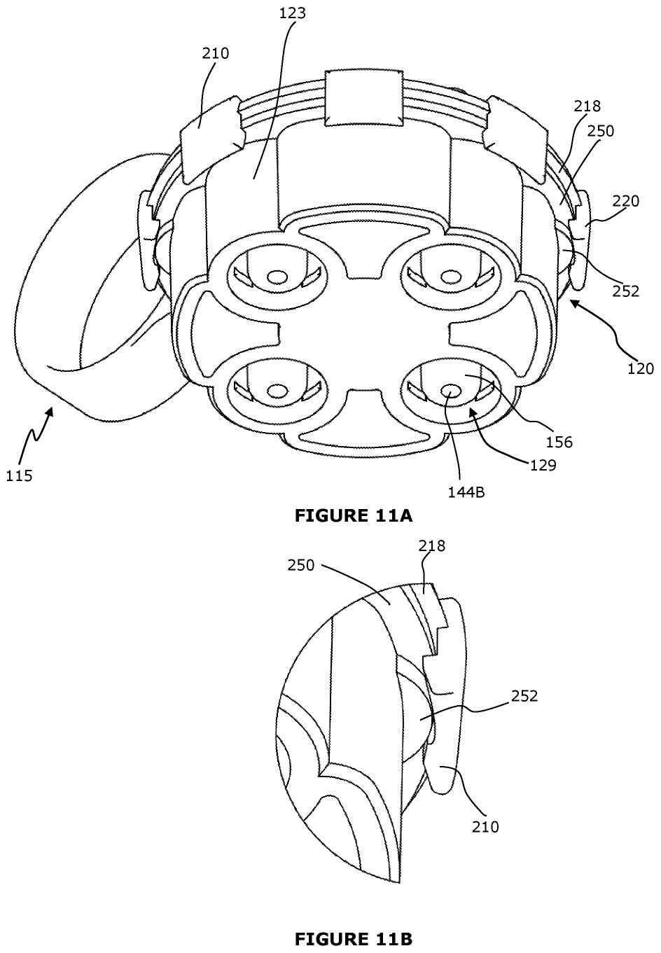

With reference to FIGS. 11 to 13, the prongs 210 of the receiving part 110 is further configured at or around its tip end portion with a recess 208 for receiving a correspondingly shaped lip projection 252 of the dispensing part 120. When the prongs 210 of the receiving part 110 is rotated into position, the lip projection 252 of the dispensing part 120 slides into the recess 208 of the prong 210 so as to frictionally couple the two parts. This also advantageously provides a haptic “click” effect so that the user knows that the parts 110, 120 are securely coupled. It is to be understood that other shapes of lip projections 252 and recesses 208 may also be suitable and used in embodiments without departing from the spirit of the invention.

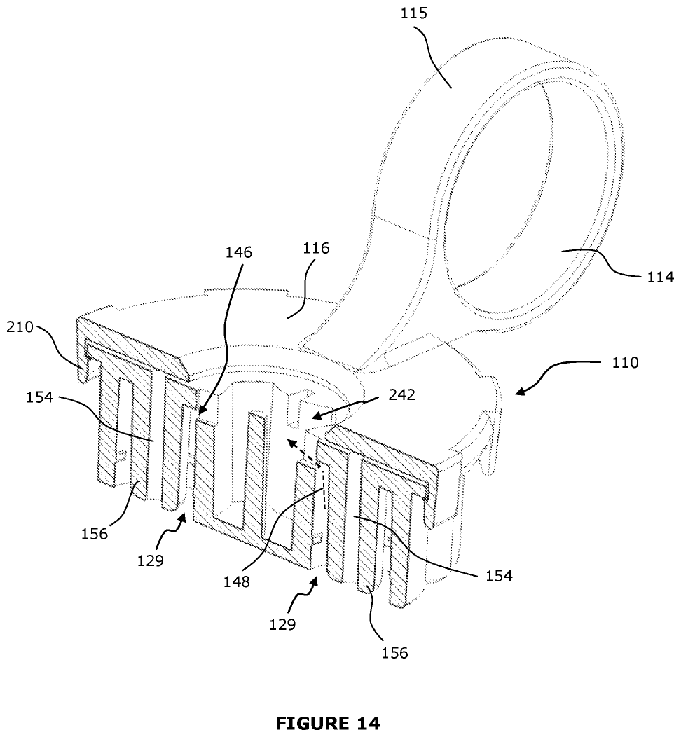

FIG. 14 shows clearly an air channel/opening 146 between the port 129 of the dispensing part 120 and the air outlet cavity 242. This opening 146 allows airflow between an internal cavity of a detachable storage container 130 and the outlet cavity 242 when the container 130 is coupled to the dispenser part 120 during use. The airflow pathway 148 allows air or any gaseous content to escape the internal cavity of the container 130 as it is being filled without causing a pressure build-up. In this embodiment, the air outlet cavity 242 is covered by an air permeable filter unit 127.

The further preferred embodiment featuring the receiving part 110 having a slightly larger diameter than the dispensing part 120 and coupling the two parts with prongs 210 is advantageously easier to manufacture, and the additional rotational friction fit interaction between the recesses 208 of the prongs 210 and the lip projections 252 of the dispensing part 120 provides users with useful haptic feedback when rotating the respective parts 110, 120.

It is to be understood that the blood collection device 100 may be used with conventional storage containers 130 and piercing apparatus (such as butterfly needle and transfer tube) as a complete blood collection system.

While various embodiments of the present invention have been described above, it should be understood that they have been presented by way of example only, and not by way of limitation. It will be apparent to a person skilled in the relevant art that various changes in form and detail can be made therein without departing from the spirit and scope of the invention. Thus, the present invention should not be limited by any of the above described exemplary embodiments.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

1. A blood collection device, comprising:
a dispenser part having a plurality of independent outlet fluid passages, each terminating at a respective outlet port for coupling with a detachable storage container; and
a receiver part having an inlet fluid passage associated with an inlet port for coupling with a blood transfer tube, the receiver part being operatively coupled with, and moveable relative to, the dispenser part so that the inlet fluid passage is moveable to align with any one of the outlet fluid passages to form a connected fluid pathway therebetween.

2. A blood collection device according to claim 1, wherein the dispenser part comprises an air channel located proximate each respective outlet port, the air channel being configured to form part of an airflow pathway between a storage container coupled to the respective outlet port and the ambient, during use.

3. A blood collection device according to claim 1, wherein the receiver part is configured to be rotatable relative to the dispenser part.

4. A blood collection device according to claim 1, wherein the receiver part is frictionally coupled to the dispenser part by way of a circumferential seal, and wherein the seal is a fluorocarbon type o-ring.

5. A blood collection device according to claim 1, wherein the receiver part comprises a handle to assist a user with moving the receiver part relative to the dispenser part in use.

6. (canceled)

7. A blood collection device according to claim 1, wherein the receiver part comprises a central cavity to allow passage of airflow.

8. A blood collection device according to claim 7, wherein the central cavity is covered with a filter unit comprising an air-permeable filter material.

9. A blood collection device according to claim 1, wherein an end of the inlet fluid passage opposing the inlet port comprises an outlet seal, and wherein the outlet seal is a fluorocarbon type o-ring.

10. A blood collection device according to claim 1, wherein the receiver part is mounted within a cavity of the dispenser part.

11. A blood collection device according to claim 1, wherein the dispenser part is in the form of a substantially round hub with downwardly-oriented outlet ports.

12. (canceled)

13. A blood collection device according to claim 1, wherein each of the respective outlet ports comprises a part-side wall of slanted geometry to assist with removal of a docked storage container by a twist-release action.

14. A blood collection device according to claim 1, wherein contact surfaces between the dispenser part and the receiver part are configured to be substantially smooth.

15. A blood collection device according to claim 1, wherein the dispenser part comprises a circumferential wall with sideway projections proximate the location of each respective outlet port.

16. (canceled)

17. A blood collection device according to claim 2, wherein the air channel leads to a central cavity of the receiver part and the airflow pathway extends out of an opening of the receiver part.

18. A blood collection device according to claim 1, wherein the receiving part is operatively coupled to the dispensing part by a plurality of prongs.

19. A blood collection device according claim 18, wherein one or more of said prongs are provided with a recess at its tip portion for receiving a corresponding lip projection of the dispensing part by frictional engagement.

20. A blood collection device according to claim 1, wherein a gasket seal having a single opening for allowing fluid flow between the inlet fluid passage and the outlet fluid passage is provided between the receiving part and the dispensing part, when assembled.

21. A blood collection system, comprising:
a blood collection device according to claim 1;
a butterfly needle connected to a blood transfer tube for coupling with the inlet port of the receiver part; and
one or more detachable storage containers for coupling with the outlet port of the dispenser part.

22. A blood collection system according to claim 21, wherein the or each detachable storage containers are not vacuum-sealed containers.

23. A blood collection system according to claim 21, wherein blood is collected from a patient during use of the system by passive venous pressure.
